(19) United States
(12) Patent Application Publication
(10) Pub. No.: US 2020/0129592 A1
(43) Pub. Date: Apr. 30, 2020

(54) COMPOSITION FOR TREATING STROKE AND METHOD FOR SCREENING THE SAME

(71) Applicants: CHA University Industry-Academic Cooperation Foundation, Pocheon-si (KR); CHA BIOTECH CO., LTD., Gangnam-gu (KR)

(72) Inventors: Ji Hwan Song, Seocho-gu (KR); Seung Hun Oh, Seongnam-si (KR); Hye Sun Kim, Yeoju-si (KR); Min Ji Lee, Songpa-gu (KR); Ji Hye Kim, Suwon-si (KR)

(73) Assignees: CHA University Industry-Academic Cooperation Foundation, Pocheon-si (KR); CHA BIOTECH CO., LTD., Gangnam-gu (KR)

(21) Appl. No.: 16/543,077

(22) Filed: Aug. 16, 2019

(30) Foreign Application Priority Data
Aug. 16, 2018 (KR) 10-2018-0095737

(51) Int. Cl. A61K 38/20; A61K 35/28; A61P 25/28

(52) U.S. Cl. CPC A61K 38/2006; A61P 25/28; A61K 35/28

(57) ABSTRACT

Provided are a composition for treating stroke and a method of screening for the same, and a pharmaceutical composition for treating stroke, the pharmaceutical composition including an IL-1 receptor antagonist as an active ingredient, and a method of screening for a therapeutic agent for stroke using the same.

### CROSS-REFERENCE TO RELATED APPLICATION

This application claims the benefit of Korean Patent Application No. 10-2018-0095737, filed on Aug. 16, 2018, in the Korean Intellectual Property Office, the disclosure of which is incorporated herein in its entirety by reference.

### BACKGROUND

### 1. Field

The present disclosure relates to a composition for treating stroke and a method of screening for the same.

### 2. Description of Related Art

According to data released by the National Statistical Office in 2010, people over 65 years of age accounted for 11.4% of Korea's population in 2011, and will reach 37.4% in 2050. It is predicted that Korea will become a super-aged society. As the population aging problem has become a social issue in recent years, the public's interest in the characteristics of the elderly population or welfare for the aged, such as housing, health, culture, leisure, etc. is increasing, and the demand for statistics is increasing. In particular, chronic degenerative diseases are emerging as more serious problems than acute infectious diseases which have been the leading cause of death for the last 50 years. Among chronic degenerative diseases, cerebrovascular disease is one of the most serious diseases and ranks as the second leading cause of death due to a single disease, and thus, there is a growing interest in cerebrovascular disease.

Such cerebrovascular disease may be largely classified into two types. One is a hemorrhagic cerebrovascular disease observed in cerebral hemorrhage, etc., and the other is ischemic cerebrovascular disease caused by the occlusion of cerebrovascular vessels, etc. Hemorrhagic cerebrovascular disease is mainly caused by traffic accidents, etc., and ischemic cerebrovascular disease mainly occurs in the elderly.

When transient cerebral infarction or cerebral hemorrhage is caused in the cerebrum, the supply of oxygen and glucose is blocked, and in neurons, ATP decreases and edema occurs, eventually leading to extensive brain damage. Neuronal cell death occurs at a considerable period of time after occurrence of cerebral infarction, and this is called delayed neuronal death. Experiments with a transient forebrain ischemic model in the Mongolian gerbil reported that delayed neuronal death is observed in the CA1 region of the hippocampus 4 days after induction of cerebral infarction for 5 minutes.

Until now, there have been two widely known mechanisms of neuronal cell death due to cerebral infarction. One is an excitotoxic neuronal death mechanism in which excess glutamate accumulates outside cells due to brain infarction, and this glutamate enters the cells and eventually causes neuronal death by accumulation of excess intracellular calcium. The other is an oxidative neuronal death mechanism which is caused by damage to DNA and the cytoplasm due to increased radicals in vivo by sudden oxygen supply during infarction-reperfusion. Based on studies of such mechanisms, much research has been conducted to search for substances that effectively suppress neuronal cell death during cerebral infarction or to reveal the mechanisms of substances. However, there are still few substances capable of effectively treating stroke.

Under this technical background, studies have been actively conducted on compositions for treating stroke, based on new molecular mechanisms (Korea Patent No. 10-1532211), but these are still inadequate.

### SUMMARY

An aspect provides a pharmaceutical composition for treating stroke, the composition including an interleukin-1 (IL-1) receptor antagonist as an active ingredient.

Another aspect provides a method of screening for a therapeutic agent for stroke, the method including measuring an expression level of an interleukin-1 receptor antagonist (IL-1RA) in a sample from a subject which is in contact with a candidate for treating stroke; and comparing the measured expression level of IL-1RA with an expression level of IL-1RA in a sample from a control subject which is not in contact with the candidate.

Still another aspect provides an IL-1 receptor antagonist including umbilical cord-derived mesenchymal stromal cells.

Still another aspect provides a cAMP-response element-binding (CREB) protein activity enhancer including umbilical cord-derived mesenchymal stromal cells.

Additional aspects will be set forth in part in the description which follows and, in part, will be apparent from the description, or may be learned by practice of the presented embodiments of the disclosure.

An aspect provides a pharmaceutical composition for treating stroke, the composition including an interleukin-1 (IL-1) receptor antagonist as an active ingredient.

As used herein, the term “treatment” means all of actions by which symptoms of stroke have taken a turn for the better or have been modified favorably by administration of the composition according to one embodiment.

“Stroke”, which is a target disease treated by administration of the composition, is also commonly called a brain attack, and refers to neurological symptoms accompanied by physical disorders such as loss of awareness, language disorder, hemiparesis, etc., which are caused by death of brain cells in the damaged area due to blockage or rupture of blood vessels which supply the blood to the brain. The stroke may include both ischemic stroke and hemorrhagic stroke.

According to an embodiment, in the acute phase of stroke, i.e., within about 24 hours after cerebral infarction or cerebral hemorrhage, administration of the IL-1 receptor antagonist may not only inhibit inflammatory responses but also contribute to recovery of nerve damage and improvement of functions. This may prevent pathological progression of stroke and minimize the sequelae associated with stroke. Therefore, the IL-1 receptor antagonist according to one embodiment may be used as an active substance for treating stroke.

In one specific embodiment, the IL-1 receptor antagonist refers to a substance that interferes with the binding of IL-1 receptor expressed in cells and IL-1, thereby attenuating some or all of its actions. The IL-1 receptor antagonist may be, for example, an umbilical cord-derived mesenchymal stromal cells, or an interleukin-1 receptor antagonist (IL-1RA) protein; in addition, it may be an antibody, an aptamer, or an antisense nucleotide against IL-1.

In one specific embodiment, the IL-1 receptor antagonist may act on inflammatory cells in stroke lesions to inhibit an IL-1 mediated inflammatory responses, wherein the inflammatory cells may be microglia or macrophages in the lesion tissue.

The pharmaceutical composition may include a pharmaceutically acceptable carrier, in addition to the active ingredient. In this regard, the pharmaceutically acceptable carrier is commonly used during formulation, and may include lactose, dextrose, sucrose, sorbitol, mannitol, starch, acacia, rubber, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil, etc. In addition, the pharmaceutically acceptable carrier may include a viral vector, a non-viral vector, a biocompatible polymer, etc. In addition to the above components, the pharmaceutically acceptable carrier may include a lubricant, a wetting agent, a sweetener, a flavoring agent, an emulsifier, a suspending agent, a preservative, etc.

The pharmaceutical composition may be administered orally or parenterally (e.g., intravenously, subcutaneously, intraperitoneally, or topically) according to a method. An administration dose may vary depending on a patient's conditions and body weight, severity of the disease, the type of the drug, administration route and time, but it may be appropriately selected by those skilled in the art.

The pharmaceutical composition may be administered in a pharmaceutically effective amount. The term “pharmaceutically effective amount” refers to an amount sufficient to treat a disease at a reasonable benefit/risk ratio applicable to any medical treatment. An effective dose level may be determined depending on factors including the type and severity of a disease of a patient, drug activity, sensitivity to a drug, administration time, administration route, discharge ratio, treatment period, and co-administered drugs, and other factors well known in the medical field. The pharmaceutical composition may be administered as a single therapeutic agent or in combination with other therapeutic agents, may be administered sequentially or simultaneously with existing therapeutic agents, and may be administered in a single dose or multiple doses. Taking all of the above factors into consideration, it is important to administer an amount such that a maximum effect may be obtained with a minimum amount without side effects, and such an amount may be readily determined by those skilled in the art.

The effective amount of the pharmaceutical composition may vary depending on a patient's age, sex, conditions, body weight, absorption of the active ingredient into the body, an inactivation rate and an excretion rate, the type of disease, and a drug used in combination, and generally, 1 mg to 500 mg per kg body weight, or a therapeutically effective amount of cells or vectors may be administered daily or every other day, or divided into 1 to 5 times a day. However, since the administration dose may be increased or decreased depending on the administration route, the severity of obesity, sex, body weight, age, etc., the administration dose does not limit the scope of the present disclosure by any method.

One aspect provides a method of treating stroke, the method including administering the pharmaceutical composition to a subject. The term “subject” means a subject in need of treatment of a disease, and more specifically, a mammal, such as a human or non-human primate, mouse, dog, cat, horse, cow, etc.

Another aspect provides a method of screening for a therapeutic agent for stroke, the method including measuring an expression level of IL-1RA in a sample from a subject which is in contact with a candidate for treating stroke; and

comparing the measured expression level of IL-1RA with an expression level of IL-1RA in a sample from a control subject which is not in contact with the candidate.

The terms or elements mentioned in the description of the screening method are the same as those mentioned above.

As used herein, the term “candidate” is a material that is expected to exhibit an effect on the treatment of stroke, for example, any substance, molecule, element, compound, or entity, or a combination thereof. For example, the candidate may include a protein, a polypeptide, a small organic molecule, a polysaccharide, a polynucleotide, etc. The candidate may also be a natural product, a synthetic compound, or a chemical compound, or a combination of two or more substances.

In the above method, “contacting” is a general meaning, and may refer to combining two or more agents (e.g., two polypeptides), combining an agent and cells (e.g., proteins and cells), etc. The contacting may occur in vitro. For example, two or more agents may be combined, or a test agent and a cell or a cell lysate and a test agent may be combined in a test tube or another container. Further, the contacting may occur in cells or in situ. For example, two polypeptides may be in contact in cells or in a cell lysate by coexpressing recombinant polynucleotides encoding the two polypeptides within cells. It is also possible to use a protein chip or a protein array in which a protein to be tested is arranged on the surface of a solid phase.

The sample may be blood, plasma, serum, urine, feces, saliva, tears, cerebrospinal fluid, cells, or tissues isolated from the subject, or a combination thereof. The sample may include a chromosome of the subject. The tissue may be a brain, a cranial nerve, or a peripheral blood vessel. The cells may be inflammatory cells, for example, microglia or macrophages.

The subject may be a mammal. In addition, the subject means including a tissue or cells isolated therefrom. The mammal may be a human, a primate, a mouse, a rat, a cow, a pig, a horse, a sheep, a dog, a cat, or a combination thereof.

The method may further include determining or selecting the candidate as a therapeutic agent for stroke, when the expression level of IL-1RA measured in the sample from the subject is increased, as compared with the expression level of IL-1RA of the non-contacted control. The change in the expression level may include the expression level of the subject which is at a similar level or shows 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900% or 1000% increase, as compared with the expression level of a non-treated control group or a negative control group.

The measuring of the expression level of IL-1RA may be performed by various methods known in the art, for example, Western blotting, dot blotting, enzyme-linked immunosorbent assay, radioimmunoassay (RIA), radioimmunodiffusion, Ouchterlony immunodiffusion, rocket

immunoelectrophoresis, immunohistochemical staining, immunoprecipitation, complement fixation assay, flow cytometry (FACS), a protein chip method, etc.

The method may further include measuring an activity level of cAMP-response element-binding (CREB) protein in a sample from a subject which is in contact with a candidate for treating stroke; and comparing the measured activity level of CREB protein with an activity level of CREB protein in a sample from a non-contacted control subject. Here, the method may further include determining or selecting the candidate as a therapeutic agent for stroke, when the activity level of CREB protein measured in the sample from the subject is increased, as compared with that of the non-treated control group. Further, for example, when the expression level of IL-1RA and the activity level of CREB protein are increased, as compared with those of the non-treated control group, the candidate may be determined or selected as a therapeutic agent for stroke.

The measuring of the activity level of CREB protein may be performed by various methods known in the art, for example, reverse transcriptase-polymerase chain reaction, real time-polymerase chain reaction, Western blotting, Northern blotting, enzyme-linked immunosorbent assay, radioimmunodiffusion, immunoprecipitation, etc.

The candidate increasing the expression level of IL-1RA and/or increasing the activity level of CREB protein in the subject sample, which is obtained through the screening method, may be an active ingredient for treating stroke. The candidate for treating stroke may act as a leading compound in a subsequent process of developing a therapeutic agent for stroke, and the structure of the leading compound may be modified and optimized to exhibit more effective therapeutic effects on stroke, thereby developing a new therapeutic agent for stroke.

Still another aspect provides an IL-1 receptor antagonist including umbilical cord-derived mesenchymal stromal cells.

In one specific embodiment, it was confirmed that the umbilical cord-derived mesenchymal stromal cells increased expression of IL-1RA in inflammatory cells, for example, microglia or macrophages, thereby playing a role as the IL-1 receptor antagonist. Therefore, the umbilical cord-derived mesenchymal stromal cells may be used in regulating molecular mechanisms in target cells, and furthermore, may be applied as an active substance for treating stroke.

Still another aspect provides a CREB protein activity enhancer including umbilical cord-derived mesenchymal stromal cells.

In one specific embodiment, it was confirmed that the umbilical cord-derived mesenchymal stromal cells increased expression of phosphorylated CREB protein in inflammatory cells, for example, macrophages, thereby playing a role as the CREB protein activity enhancer. Therefore, the umbilical cord-derived mesenchymal stromal cells may be used in regulating molecular mechanisms in target cells, and furthermore, may be applied as an active substance for treating stroke.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows results of measuring levels of VEGF, TGF-β1, HGF, and IDO secreted by umbilical cord-derived mesenchymal stem cells (hMSCs) according to one embodiment;

FIGS. 2A and 2B show functional changes in stroke animal models according to dosage and time points of administration of hMSCs, in which FIG. 2A shows results of a behavioral test and FIG. 2B shows results of measuring changes in the infarct size;

FIGS. 3A and 3B show therapeutic effects in stroke animal models according to intravenous administration of hMSCs, in which FIG. 3A shows results of a behavioral test and FIG. 3B shows results of measuring changes in the infarct size;

FIG. 4 shows TUNEL assay results of examining neuronal cell death inhibitory effects in stroke animal models according to intravenous administration of hMSCs;

FIGS. 5A to 5C show immunohistochemical analysis results of examining pathological changes in the infarct brain tissue in stroke animal models according to intravenous administration of hMSCs, in which FIG. 5A shows results of comparing the numbers of ED-1-positive cells and Iba-1-positive cells, FIG. 5B shows results of comparing proportions of iNOS cells in ED-1-positive cells, and FIG. 5C shows results of comparing the number of ELANE positive cells;

FIG. 6 shows myeloperoxidase (MPO) ELISA results of examining pathological changes in the infarct brain tissue in stroke animal models according to intravenous administration of hMSCs;

FIGS. 7A to 7C show polymerase chain reaction (PCR) results of examining changes in inflammatory cytokine expression in the infarct brain tissue in stroke animal models according to intravenous administration of hMSCs, in which

FIG. 7A shows results of comparing changes in IL1B expression, FIG. 7B shows results of comparing changes in TNF expression, and FIG. 7C shows results of comparing changes in MMP9 expression;

FIG. 8 shows results of examining changes in gene expression in the infarct brain tissue in stroke animal models according to intravenous administration of hMSCs;

FIGS. 9A and 9B show results of quantitatively comparing changes in gene expression in the brain tissue in stroke animal models according to intravenous administration of hMSCs, in which FIG. 9A shows results of comparing changes in IL1RN mRNA expression, and FIG. 9B shows results of comparing changes in IL-1ra protein expression;

FIGS. 10A and 10B show results of examining cell subpopulations that contribute to IL-1ra upregulation in stroke animal models according to intravenous administration of hMSCs, in which FIG. 10A shows immunohistochemical analysis results of examining IL-1ra positive cells among ED-1-positive cells, and FIG. 10B shows results of quantitatively comparing proportions of IL-1ra-positive cells among D-1-positive cells;

FIGS. 11A and 11B show changes in inflammatory cytokine expression by co-treatment of Raw 264.7 cells with LPS and hUMSCs-CM, in which FIG. 11A shows results of examining changes in IL-1β expression, and FIG. 11B shows results of examining changes in IL-1ra expression;

FIGS. 12A to 12C show the relationship between hUMSCs-mediated IL-1ra expression and cAMP-response element-binding (CREB) protein in Raw 264.7 cells, in which FIG. 12A shows Western blotting results of examining p-CREB protein expression, etc., FIG. 12B shows results of quantitatively comparing p-CREB protein expression, and FIG. 12C shows results of quantitatively comparing p-NF-κB expression;

FIG. 13 shows immunohistochemical analysis results of examining changes in p-CREB and p-NF-κB expression by co-treatment of Raw 264.7 cells with LPS and hUMSCs-CM;

FIG. 14 shows results of examining changes in IL-1ra expression by co-treatment of Raw 264.7 cells with hUMSCs-CM and CREB inhibitor (KG501);

FIG. 15A and 15B show results of examining changes in IL-1ra expression after transfection of Raw 264.7 cells with CREB siRNA (siCREB); and

FIG. 16 shows results of examining changes in URN expression by treatment with hUMSCs-CM, after transfection of LPS-stimulated Raw 264.7 cells with CREB siRNA (siCREB).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### REFERENCE EXAMPLE 1

### Experiment Preparation and Experiment Process

(1) Preparation of hUMSCs and Characterization Thereof

With informed consent from a healthy donor, umbilical cord-derived mesenchymal stem cells (hUMSCs) were retrieved from the umbilical cord stored at CHA Bundang Medical Center (Seongnam, Korea). Preparations of hUMSCs were conducted in the GMP facility, and isolation and expansion of hUMSCs were performed according to the Good Clinical Practice (GCP) guidelines of the Master Cell Bank. First, after umbilical vessels were removed from the retrieved hUMSCs, Wharton's jelly was sliced into 1-5-mm explants to isolate hUMSCs. Isolated hUMSCs were attached to a culture plate containing α-MEM (HyClone, IL) supplemented with 10% FBS (HyClone, IL), FGF4 (R&D Systems, MN), and heparin (Sigma-Aldrich, MO), and subsequently cultured. The medium was changed every 3 days. After 15 days, the umbilical cord fragments were discarded, and the hUMSCs were passaged with TrypLE (Invitrogen, MA), and expanded until they reached sub-confluence (80% to 90%). The hUMSCs were incubated under hypoxic conditions (3% O₂, 5% CO₂, and 37° C.). The hUMSCs at passage 7 were used in the present experiment.

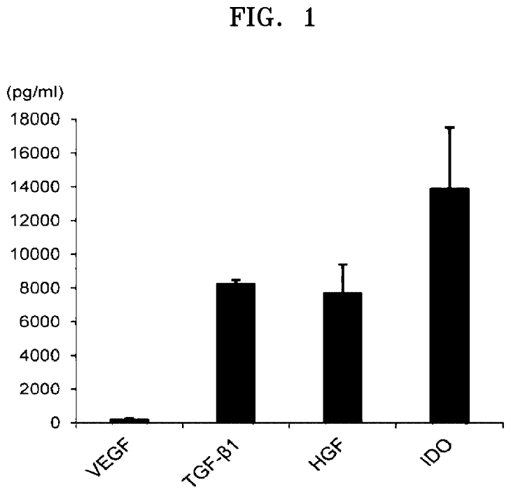

Thereafter, karyotype analysis confirmed that the hUMSCs contained a normal human karyotype. Further, using reverse transcriptase polymerase chain reaction, the absence of viral pathogens (human immunodeficiency virus-1 and 2, cytomegalovirus, hepatitis B virus, hepatitis C virus, human T-lymphocytic virus, Epstein-Barr virus, and mycoplasma) in cell pellets was confirmed. Fluorescence-activated cell sorting (FACS) analysis was performed as previously described to identify the immunophenotype of hUMSCs. The hUMSCs expressed high levels of cell surface markers for MSCs (CD44, CD73, CD90, and CD105), but expression of markers for hematopoietic stem cells (CD31, CD34, and CD45) and HLA-DR was negligible. When hUMSCs (n=3) reached 100% confluence, they were further cultured in serum-free medium for 48 hrs, and hUMSCs-CM was collected therefrom. Subsequently, protein concentrations of TGF-β1 (Human TGF-β1 ELISA kit, R&D Systems, MN), VEGF (Human VEGF ELISA kit, R&D Systems, MN), HGF (Human HGF ELISA kit, Cloud-Clone Corp., TX), and IDO (Human IDO ELISA kit, BlueGene Biotech., Shanghai, China) derived from hUMSCs-CM were measured using commercially available enzyme-linked immunosorbent assay (ELISA) kits according to the manufacturer's instructions . . . As a result, as shown in FIG. 1, the hUMSCs secreted high levels of TGF-β1, HGF, and IDO. These experimental data indicate that hUMSCs have characteristics of MSCs and secrete cytokines and trophic factors that are involved in the immune response and tissue repair.

(2) Construction of Stroke Animal Model

In this experiment, a total of 151 male Sprague-Dawley rats weighing 270 g to 300 g were used, and middle cerebral artery occlusion (MCAo) was induced in the rats by a method as reported by Longa et al.

(3) Statistical Analysis and Ethics statements

Statistical analyses were performed using Statistical Analysis System program (Enterprise 4.1; SAS Korea) and MedCalc statistical software (MedCalc software, ver. 11.6, Mariakerke, Belgium). The statistical significance between two groups in the histological or infarct size measurements was analyzed by the Mann-Whitney U test. The statistical significance of multiple comparisons for real-time PCR or ELISA was analyzed using the Kruskal-Wallis test with a post hoc Conover's test for pairwise comparisons of subgroups. The analysis of functional tests was performed using the two-way mixed analysis of variance (mixed ANOVA) test. Statistical significance was considered at p<0.05 and p<0.001, and all values are presented as the means±standard error of the mean (SEM). The statistical analysis of microarray data was evaluated by the same method as in the previous experiment.

Further, this experiment was approved by the Institutional Review Board at the CHA Bundang Medical Center for the use of umbilical cord (IRB no.: BD2013-004D). All experimental animals were manipulated in accordance with guidelines provided by the Institutional Animal Care and Use Committee of CHA University.

### EXPERIMENTAL EXAMPLE 1

### Identification of Effects of hUMSCs on Neuronal damage reduction and functional improvement in stroke animal model

In this Experimental Example, behavioral tests, infarct size tests, and TUNEL were performed to investigate effects of intravenous administration of hUMSCs (IV-hUMSCs) on neuronal damage reduction and functional improvement in the acute phase of cerebral infarction.

(1) Intravenous Administration of hUMSCs

To test functional changes according to dosage and time points of administration of IV-hUMSCs, the above-described stroke animal models (MCAo-induced rats) were randomly divided into five groups according to the dose and time points of administration of hUMSCs.

Group 1 (G1): rats administered with saline at 24 hrs post-MCAo induction

Group 2 (G2): rats administered with 1×10⁵ IV-hUMSCs at 24 hrs post-MCAo induction

Group 3 (G3): rats administered with 5×10⁵ IV-hUMSCs at 24 hrs post-MCAo induction

Group 4 (G4): rats administered with 1×10⁶ IV-hUMSCs at 24 hrs post-MCAo induction

Group 5 (G5): rats administered with 1×10⁶ IV-hUMSCs at 7 days post-MCAo induction

hUMSCs mixed with 500 μl of saline were administered into tail veins of the corresponding group for 5 min at each time point of administration (24 hrs or 7 days post-MCAo induction). No profound bleeding occurred during administration, and vital signs in all rats were stable during the procedure. All rats were injected with cyclosporine A (5 mg/kg) intraperitoneally from the previous day of administration and up to 8 weeks after cell administration.

Further, after testing functional changes according to the dosage and time points of administration of IV-hUMSCs, another independent experiment was performed to confirm therapeutic effects of IV-hUMSCs administration. Over a total of 4 weeks after induction of cerebral infarction, 1×10⁶ IV-hUMSCs were administered at 24 hrs after MCAo (IV-hUMSC group, n=10), and a control group was administered with saline (saline group, n=10).

(2) Behavioral Test

Behavioral tests were conducted by investigators who were blinded to each group, and a rotarod test and an mNSS test were performed as previously described. For the rotarod test, each rat was pre-trained three times a day for 3 days before MCAo induction to minimize variation among animals. The rat was placed on the rotarod wheel to record the time of endurance on the wheel. A rod speed of the rotarod device was gradually increased from 4 rpm to 40 rpm for 2 min. Thereafter, the time that was taken for each rat to fall down from the rotating wheel was recorded, and the average time was calculated from a total of three trials. The test was conducted 1 day before MCAo (pre), on the day (DO) of MCAo induction, and 2 days (D2) after MCAo induction. Afterward, the test was conducted once per week over a total of 8 weeks.

For the modified neurological severity score (mNSS) test, each rat was tested 1 day after MCAo induction, and up to a total of 8 weeks after cell administration. The rat was given a score, which was the sum of the individual neurological test scores. A high score represents the most severe condition, whereas a low score represents the normal condition.

(3) Measurement of Infarct Size and TUNEL Assay

At 8 weeks post MCAo, cresyl violet staining was used to measure the infarct volume in the MCAo models (n=7 for each group). In an independent in vivo experiment group, the infarct size was compared at 72 hrs post MCAo (48 hours after IV-hUMSC administration) using 2,3,5-triphenyltetrazolium chloride between the IV-hUMSC- and saline-administered groups (n=5 for each group). The detailed tissue preparation methods were performed as described previously. Thereafter, the infarct size was estimated as a percentage of the intact contralateral hemisphere by the following [Equation 1]:

Estimated infarct size (%)=[1−(area of remaining ipsilateral hemisphere/area of intact contralateral hemisphere)]×100. [Equation 1]

The areas of interest were measured with ImageJ software (ImageJ, National Institutes of Health), and the measured values represent summed results for six serial coronal sections per brain.

TUNEL assay for cell death was performed as described previously. The subject tissue was counterstained with 4′,6-diamidine-29-phenylindole dihydrochloride (DAP6I) which is a nuclear marker. Fluorescently labeled specimens were observed under a confocal laser-scanning microscope (LSM510, Carl Zeiss Microimaging Inc., Munchen, Germany).

(4) Experimental Results

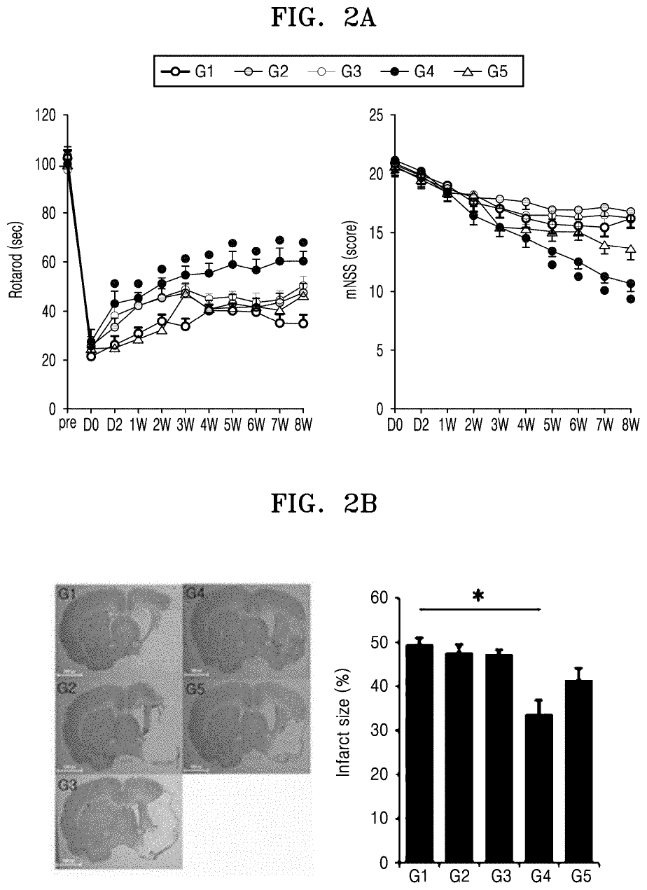

As a result of evaluating functional changes according to the dosage and time points of administration of IV-hUMSCs, in the rotarod test and the mNSS test, significant improvement in neuronal functions was observed in rats administered with a dose of 1×10⁶ hUMSCs (G4 group), as compared with G1 group (saline-administered group) which is the control group, as shown in FIG. 2A. Further, in the test of the infarct size, significant reduction was observed in G4 group, as compared with G1 group (G4 vs G1: 33.6±3.3% vs. 49.4±1.5%, p=0.004), as shown in FIG. 2B. However, even though administered with the same dose of hUMSCs as in G4 group, they did not show any significant effects in the functional tests in the delayed phase of stroke (G5 group).

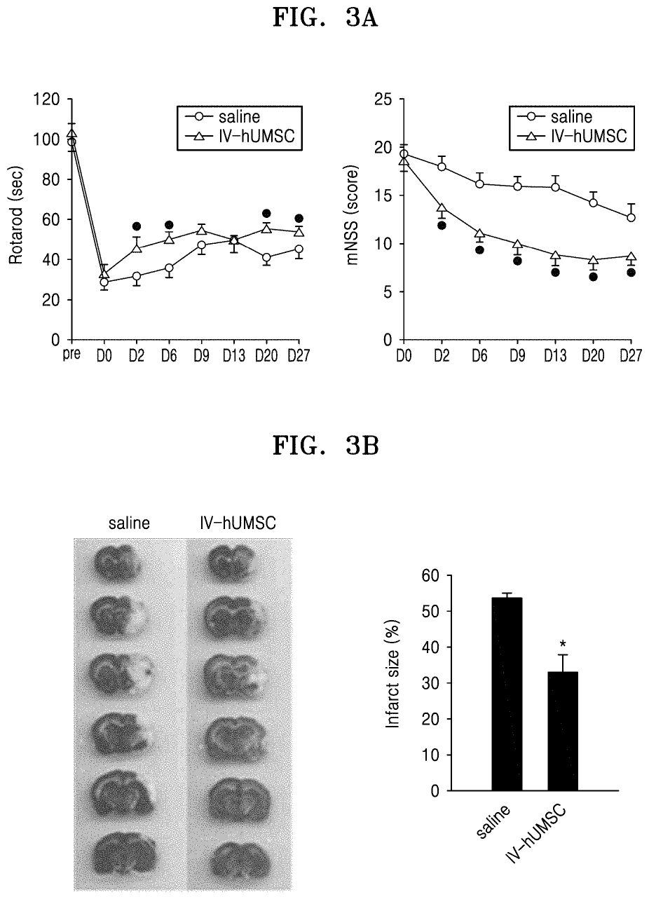

Further, as a result of examining therapeutic effects by administration of IV-hUMSCs, rats (IV-hUMSC group) administered with a dose of 1×10⁶ IV-hUMSCs at 24 hrs post-MCAo induction showed a significant improvement over 4 weeks of functional tests, and had a remarkably small infarct size at 72 hrs post-MCAo induction, as shown in FIGS. 3A and 3B.

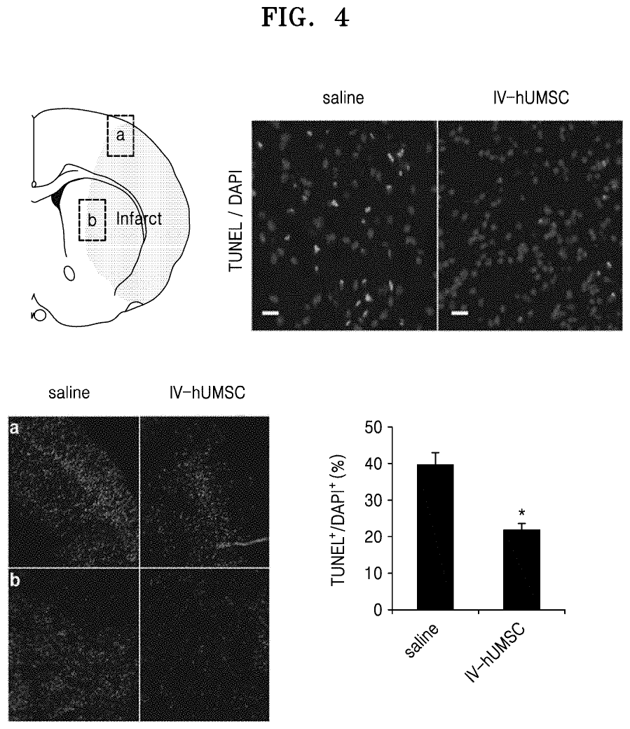

Lastly, as a result of performing terminal deoxynucleotidyl transferase dUTP nick-end labeling (TUNEL) assay to investigate the effects of IV-hUMSCs on neuronal cell death, there were numerous TUNEL-positive cells in the peri-infarct area of the saline-treated group, and extensive neuronal cell death was observed, whereas the IV-hUMSC-administered group showed the significantly low number of TUNEL-positive cells in the peri-infarct area at 72 hours post-MCAo induction, as compared with the saline-treated group (IV-hUMSCs vs saline: 22.1±1.9% vs 39.5±3.8%, p=0.006), as shown in FIG. 4.

Therefore, the results of a series of experiments indicate that intravenous administration of the cerebral infarct tissues in the acute phase with about 1×10⁶ hUMSCs may give rise to a significant functional improvement as well as a reduction of damaged area, i.e., infarct size.

Meanwhile, in the following Experimental Example, biochemical and/or histological analyses were conducted for rats (IV-hUMSC) intravenously administered with 1×10⁶ hUMSCs at 24 hrs post-MCAo induction, and rats treated with saline alone (saline group) as a control.

### EXPERIMENTAL EXAMPLE 2

### Identification of Inflammation-Attenuating Effects of hUMSCs in Acute Phase of Cerebral Infarction

In this Experimental Example, immunohistochemical analysis and real-time PCR were performed to examine the inflammation-attenuating effects according to intravenous administration of hUMSCs (IV-hUMSCs).

(1) Immunohistochemical Analysis and MPO Analysis

Pathological changes in the cerebral infarct tissue were examined by performing immunohistochemical analyses at 72 hrs post-MCAo induction (n=5 for each group). Immunohistochemical markers, such as those for macrophages/microglia (Iba-1, iNOS, and CD206) and neutrophils (ELANE), were used to evaluate changes in the related factors in the cerebral infarct tissue after IV-hUMSC administration. Further, myeloperoxidase (MPO) was measured in the supernatants of rat brain tissue using an MPO assay kit (Hycult Biotech, Uden, the Netherlands). The ELISA procedure was conducted according to the manufacturer's instructions. Independent experiments were duplicated on different days.

(2) Real-Time Polymerase Chain Reaction

At 72 hrs post-MCAo induction, real-time PCR was performed for MCAo-treated ipsilateral hemisphere to examine changes in inflammatory cytokines related to the stroke pathophysiology, including interleukin-1β coding gene (IL1B). TNF-α coding gene (TNF), and matrix metalloproteinase 9 coding gene (MMP9). In this experiment, RNA from normal rat brains as well as saline-treated MCAo rat brains as controls were used to investigate changes in inflammatory gene expression in cerebral infarct tissues according to IV-hUMSC administration. Total RNAs were reverse-transcribed to the complementary DNA strand using a SuperScript® II First-Strand Synthesis System (Invitrogen, MA). Expression of mRNAs was quantified using a CFXTM real-time system (Bio-Rad Laboratories, CA) and a Quantitect® SYBR Green PCR kit (Qiagen, Hilden, Germany). The real-time PCR was duplicated for each gene, and the mean value thereof was used for the statistical analysis. The mRNA levels of selected genes were normalized to GAPDH. The fold difference is represented as a 2^{-ddCT} value that was calculated by the comparative threshold (CT) cycle method.

(3) Experimental Results

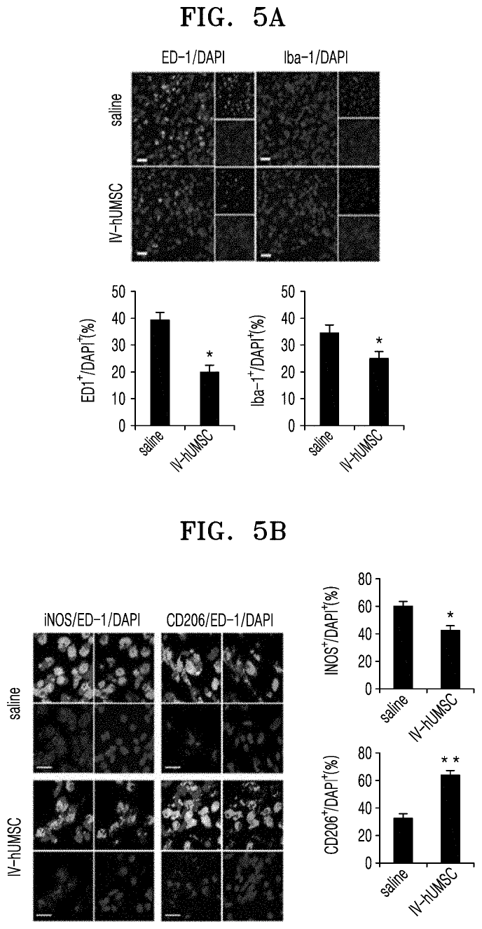

As a result of immunohistochemical analysis, in the control group (saline-administered group), numerous ED-1 (rat homolog of human CD68)-positive cells and ionized calcium-binding protein adapter molecule 1 (Iba-1)-positive cells were found in the peri-infarct area at 72 hrs post-MCAo induction, however, in the IV-hUMSC-administered group, the numbers of ED-1-positive cells (IV-hUMSCs vs saline: 20.2±2.1% vs 39.3±2.9%, p=0.006) and Iba-1-positive cells (IV-hUMSCs vs saline: 25.6±2.1% vs. 34.9±2.9%, p=0.017) were significantly reduced, as shown in FIG. 5A. Further, a proportion of inducible nitric oxide synthase (iNOS)-positive cells in ED-1-positive cells was lower in the IV-hUMSC-administered group than in the control group (IV-hUMSCs vs saline: 43.7±4.3% vs 60.3±5.1%, p=0.003), whereas a proportion of CD206-positive cells in ED-1-positive cells was higher in the IV-hUMSC-administered group than in the control group (IV-hUMSCs vs saline: 66.5±3.3% vs 34.1±4.3%, p<0.001), as shown in FIG. 5B. Furthermore, changes in neutrophils infiltrated into the infarct area after IV-hUMSC administration was evaluated by neutrophil elastase (ELANE) immunostaining. As a result, significantly fewer ELANE-positive cells were observed in the IV-hUMSC-administered than in the control group (IV-hUMSCs vs saline: 4.9±0.9% vs 16.7±1.9%, p=0.001), as shown in FIG. 5C.

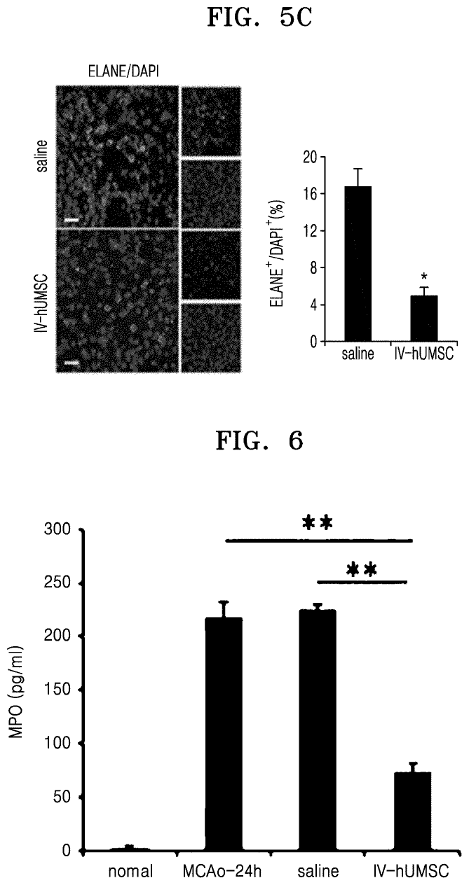

Next, the results of the myeloperoxidase (MPO) ELISA indicated that the level of MPO at 72 hrs post-MCAo induction was lower in the IV-hUMSC-administered group than in the control group (IV-hUMSCs vs. saline: 74.1±4.8 pg/ml vs 225.1±4.7 pg/ml, p<0.001), as shown in FIG. 6.

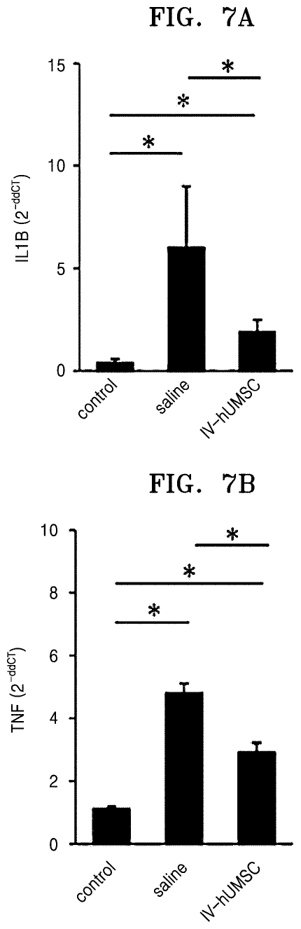

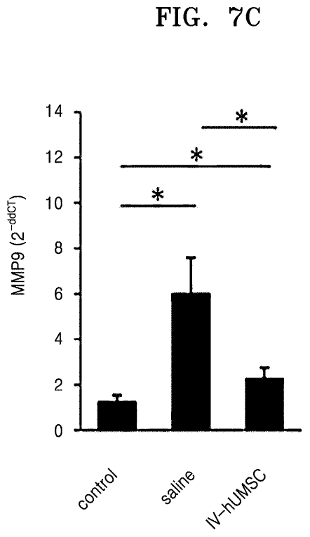

Lastly, real-time polymerase chain reaction (PCR) analysis of inflammation-related genes revealed that upregulation of IL1B, TNF, and MMP9 expression was observed in cerebral infarct tissues, but this was remarkably reduced in the IV-hUMSC-administered group, as shown in FIG. 7.

Therefore, the results of a series of experiments indicate that intravenous administration of hUMSCs into the cerebral infarct tissue in the acute phase may contribute to alleviation of inflammation at the corresponding site.

### EXPERIMENTAL EXAMPLE 3

### Changes in Endogenous IL-1ra Expression in Cerebral Infarct Tissue in Acute Phase

In this Experimental Example, microarray analysis and real-time PCR were performed to investigate factors closely related to the therapeutic effect of intravenous administration of hUMSCs (IV-hUMSCs).

(1) Microarray Analysis

At 72 hrs post-MCAo induction, the ipsilateral hemisphere subjected to MCAo was used for mRNA microarray analysis. RNA was isolated as quickly as possible from the ipsilateral hemisphere in non-MCAo-induced rats (control group, n=5), rats (n=6) administered with 1×10⁶ IV-hUMSCs at 24 hrs post-MCAo induction, and rats administered with saline at 24 hrs post-MCAo induction by homogenization with TRIzol® reagent (Thermo Fisher Scientific, MA) and RNeasy columns (Qiagen, Hilden). In addition to the sham control, the present inventors also used normal rat brains without MCAo induction as an additional control to investigate the changes in inflammatory gene expression after treatment with IV-hUMSCs in cerebral infarct tissues. To ensure RNA quality, only samples with an optical density of 260 nm/280 nm ratio above 1.08 with an Agilent 2100 Bioanalyzer (Agilent Technologies, CA) were used for microarray analysis. RNA labeling and purification were performed, and the samples were hybridized to Agilent rat mRNA microarray chips (SurePrint G3 Rat Gene Expression 8×60 k, Agilent Inc., CA) according to the manufacturer's instructions. The array was scanned using Agilent Technologies G2600DSG12494263 (Agilent Inc., CA). Array data exporting processing and analysis were performed using Agilent Feature Extraction software (v100.0.1.1). The data were filtered by log transformation and quantile normalization. The array data were statistically analyzed using the Student's t test with false discovery rate correction (Benjamini-Hochberg test) for pairwise comparisons among each group. A differentially expressed transcript was described as a gene with a more than two-fold difference (FD) and significant difference in the corrected p value (p)<0.01 for consideration of the multiple-comparison hypothesis. All data analyses and visualization of differentially expressed transcripts were conducted using R 3.0.1 (www.r-project.org). The microarray data were registered in the GEO repository (accession no. GSE78731).

(2) Immunohistochemical Assay, etc.

To identify the cell subpopulations that contribute to IL-1ra upregulation, a double immunochemical study was performed using antibodies against IL-1ra and ED-1 (microglial markers), NeuN (a neuronal marker), or Reca-1 (an endothelial cell marker) in cerebral infarct tissues. Further, at 72 hrs post-MCAo induction, the ipsilateral hemisphere subjected to MCAo was subjected to real-time PCR to examine expression of IL-1-mediated inflammation regulators, i.e., IL1RN and IL-1ra. Meanwhile, detailed experiments were conducted in the same manner as in (1) and (2) of Experimental Example 2.

(3) Experimental Results

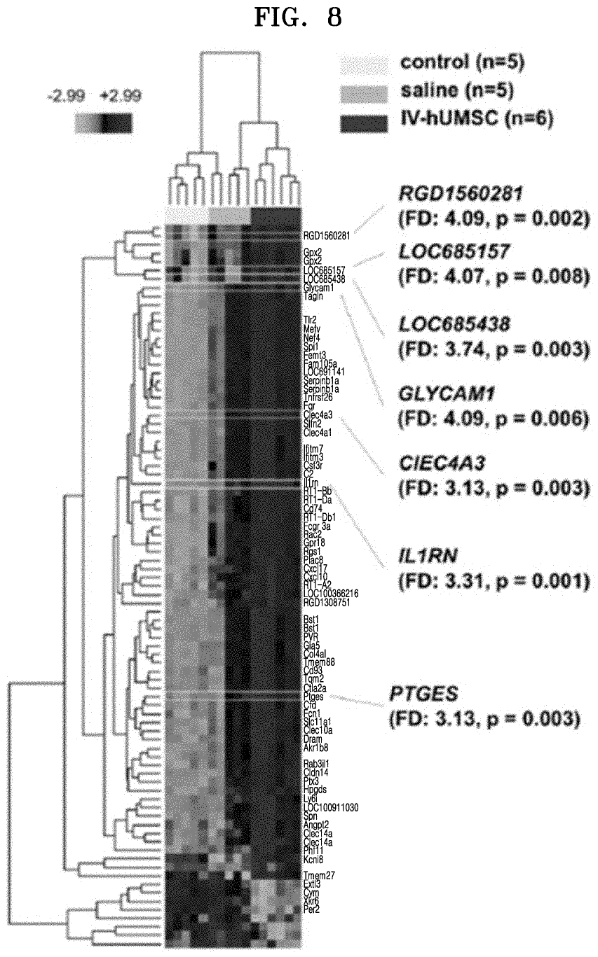

mRNA microarray was performed for brain tissues derived from the IV-hUMSC-administered group and the saline-administered group at 72 hrs post-MCAo induction. As a result, comparison of gene expression between the control group and the saline group at 72 hrs post-MCAo induction showed that a total of 595 transcripts (among them, 553 transcripts were upregulated and 42 transcripts were downregulated) were differentially expressed in the IV-hUMSC-administered group, as shown in FIG. 8. When gene expression profiles were compared between the IV-hUMSC-administered group and the saline group, a total of 85 transcripts (among them, 77 transcripts were upregulated and 8 transcripts were downregulated) were differentially expressed. Among them, IL1RN which is a gene encoding the interleukin-1 receptor antagonist (IL-1ra) was one of the most strongly upregulated genes in the IV-hUMSC-administered group, as compared with the saline-administered group.

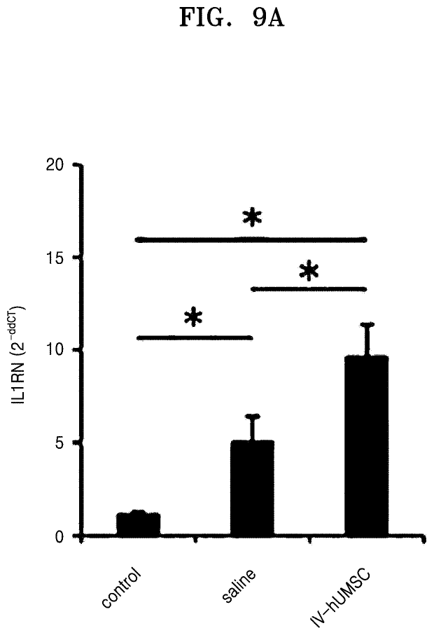

Meanwhile, IL-1ra is a natural antagonist of IL-1 and is known to play a role in regulating IL-1-mediated inflammation in cerebral infarct tissues. Thus, based on the above experimental results, it was hypothesized that the therapeutic effect of IV-hUMSCs on cerebral infarction is mediated by IL-1ra, and changes in IL1RN mRNA and IL-1ra protein expression were examined in cerebral infarct tissues after IV-hUMSC administration. As a result, IL1RN mRNA was upregulated by IV-hUMSC administration after MCAo induction, and thus IL-1ra protein levels were significantly increased (IV-hUMSCs vs saline: 237.5±49.0 pg/ml vs 119.6±15.6 pg/ml, p=0.01), as shown in FIG. 9.

The above experimental results indicate that IL-1ra is closely related with the therapeutic effect of intravenous administration of hUMSCs (IV-hUMSCs).

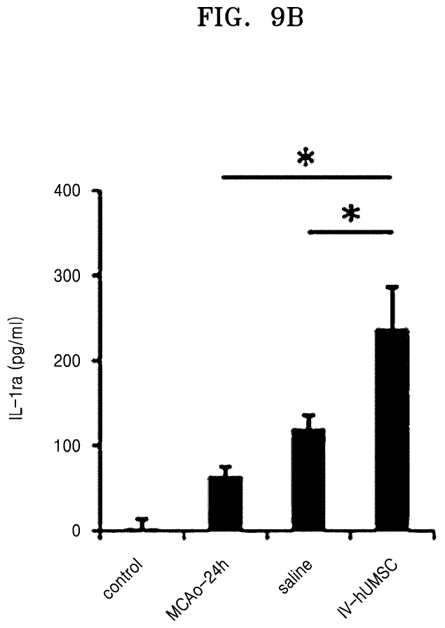

Further, to identify the cell subpopulations that contribute to IL-1ra upregulation, immunohistochemical assay was performed. As a result, it was observed that a proportion of IL-1ra-positive cells in ED-1-positive cells was significantly higher in the IV-hUMSC-administered group than in the saline-administered group (IV-hUMSCs vs saline: 34.8±2.5% vs 22.1±3.4%, p=0.01), as shown in FIG. 10. In particular, IV administered hUMSCs were hardly detectable in the cerebral infarct tissues (less than 1% of administered cells) at 72 hrs and 4 weeks post-MCAo induction by immunostaining using a human-specific nuclear antibody. In the ELISA experiment, IL-1ra was not detected in the conditioned medium of hUMSCs (hUMSCs-CM) (<3.2 pg/ml).

The results of a series of experiments suggest that upregulation of IL-1ra according to intravenous administration of hUMSCs (IV-hUMSCs) was originated from inflammatory cells in the cerebral infarct tissues, such as microglia and macrophages, rather than from the transplanted cells.

### EXPERIMENTAL EXAMPLE 4

### Changes in CREB Activity and IL-1ra Release in Macrophage by hUMSCs

(1) Treatment of Raw 264.7 Cells with Conditioned Medium of hUMSCs

A mouse macrophage cell line (Raw 264.7 cell, ATCC, VA) was cultured according to the manufacturer's instructions. The raw 264.7 cells were treated with either LPS (100 ng/ml, Sigma-Aldrich, MO) or LPS together with hUMSCs-CM for 24 hrs, and the supernatants were isolated from each treated group.

(2) Western Blot Analysis

After homogenization of Raw 264.7 cells, proteins were isolated using a protein lysis buffer (PRO-PREP™, Intron Biotechnology, Seongnam, Republic of Korea) and subjected to immunoblot analysis according to the manufacturer's instructions. Whole proteins were separated on SDS-PAGE gels and immunoblotted using primary antibodies. The primary antibodies used are as follows: (1) anti-CREB and anti-p-CREB (1:1000, Cell Signaling Technology, MA); (2) anti-NF-κB p65 and anti-p-NF-κB p65 (1:1000, Santa Cruz Biotechnology, TX); and (3) anti-IL-1ra(1:500, Santa Cruz Biotechnology, TX). GAPDH (1:5000, Santa Cruz Biotechnology, TX) was used as an internal control. Quantification of the bands was performed using an NIH ImageJ program. Independent experiments were performed in triplicate on different days.

(3) Inhibition of p-CREB

Raw 264.7 cells (2×10⁵) were seeded onto the plate, and incubated for 24 hrs. The cells were pretreated with a serum-free medium containing KG501 (2 μM, 5 μM, and 10 μM, Sigma-Aldrich, MO) for 45 min. The supernatant was then removed from the dish, and the cells were treated with hUMSCs-CM in the presence of LPS (200 ng/ml). The supernatant was used for IL-1ra ELISA analysis. Independent experiments were triplicated on different days.

(4) Knockdown of CREB

After Raw 264.7 cells (2×10⁵) were plated in 6-well plates, transfection with 100 nM of CREB siRNA (siCREB) and control (siCtr) was performed for 48 hrs using Lipofectamine 3000 reagent (Invitrogen, MA) according to the manufacturer's instructions. The siCREB (sense: 5′-CCACAAAUCAGAUUAAUUUUU-3″, antisense: 5′-AAAUUAAUCUGAUUUGUGGUU-3″) and siCtr (sense: 5-ACGUGACACGUUCGGAGAA-3′, antisense: 5′-UUCUCCGAACGUGUCACGU-3″) were purchased from Genolution Pharmaceuticals, Inc. After transfection, the RNA and proteins were isolated and used for PCR and western blotting, respectively.

(5) Enzyme-Linked Immunosorbent Assay (ELISA)

IL-1β and IL-1ra levels were measured in supernatants of Raw 264.7 cells using commercially available ELISA kits (IL-1β Quantikine ELISA and IL-1 ra Quantikine ELISA kits, R&D Systems, MN).

(6) Experimental Results

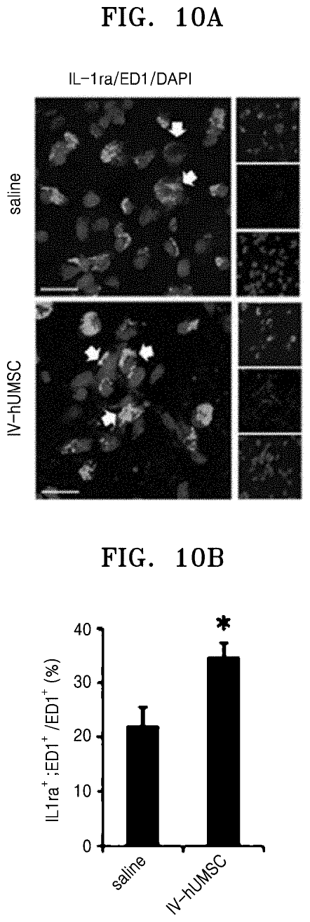

In addition to microglia in the central nervous system, circulating macrophages also play a role in inflammatory response caused post-cerebral infarction by infiltrating into brain parenchyma and releasing pro-inflammatory cytokines. It is known that MSCs polarize circulating macrophages into anti-inflammatory phenotype and recruit a number of inflammatory cells that contribute to healing of the damaged tissue. Thus, the effect of hUMSCs-CM on IL-1ra expression in a mouse macrophage cell line (Raw 264.7 cells) was investigated. First, treatment with lipopolysaccharide (LPS) strongly elevated the levels of both IL-1β and IL-1ra in Raw 264.7 cells. Thus, it was expected that IL-1ra expression was upregulated by NF-κB signaling involved in inflammatory responses under the inflammatory milieu, such as IL-1β activation and a compensatory mechanism. However, as shown in FIG. 11, when Raw 264.7 cells were co-treated with LPS and hUMSCs-CM, the IL-1β level was reduced, whereas the IL-1ra level was rather increased to show a totally different pattern. This result suggests that mechanisms other than NF-κB are involved in the upregulation of IL-1ra according to treatment with hUMSCs-CM.

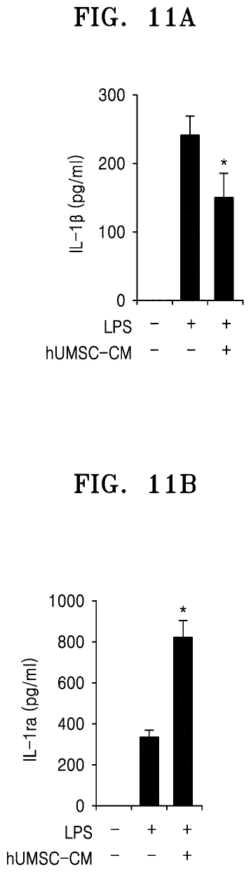

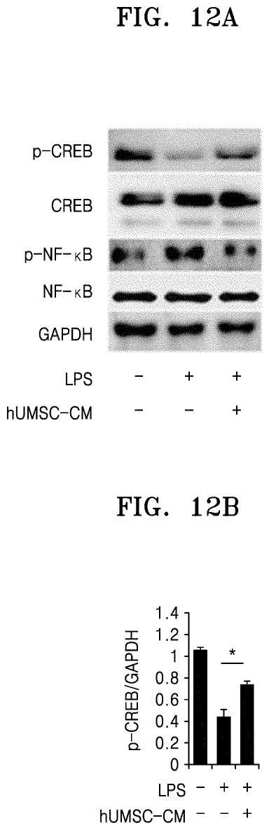

Therefore, the effect of cAMP-response element-binding protein (CREB) in hUMSCs-mediated IL-1ra expression in macrophages was further investigated. As shown in FIG. 12, the results of Western blot analysis showed that expression of phosphorylated CREB (p-CREB) proteins was significantly increased but phosphorylated NF-κB (p-NF-κB) was decreased in Raw 264.7 cells when co-treated with hUMSCs-CM and LPS. Further, as shown in FIG. 13, the results of immunocytochemical assay of p-CREB and p-NF-κB showed that co-treatment with LPS and hUMSCs-CM strongly increased expression of p-CREB in Raw 264.7 cells, as compared with the treatment with LPS alone.

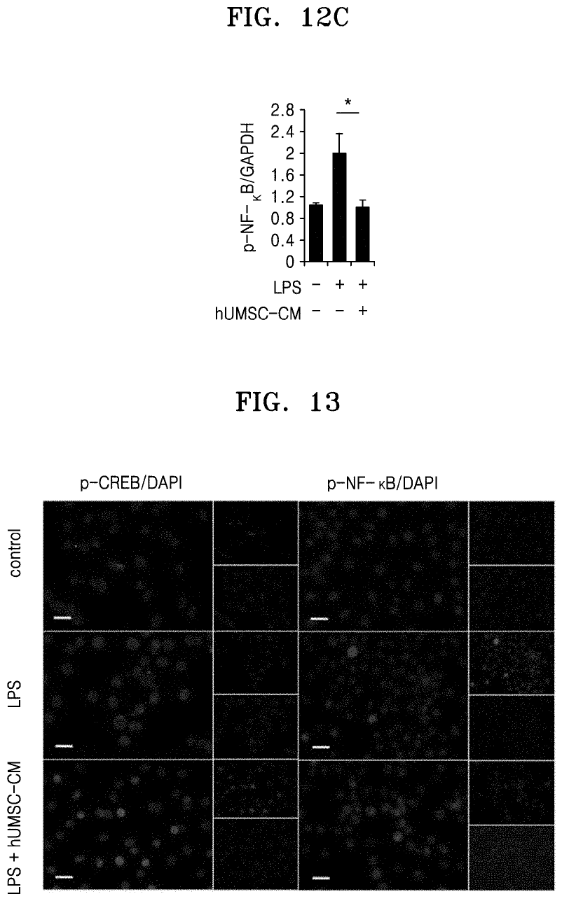

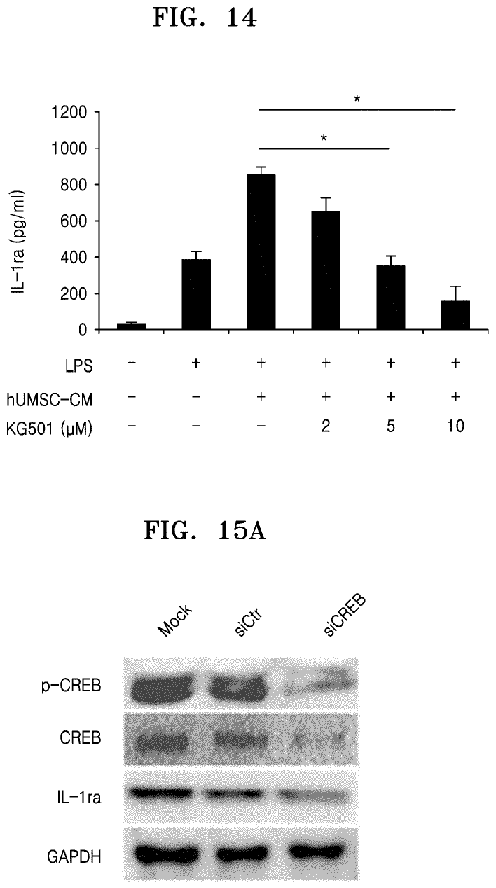

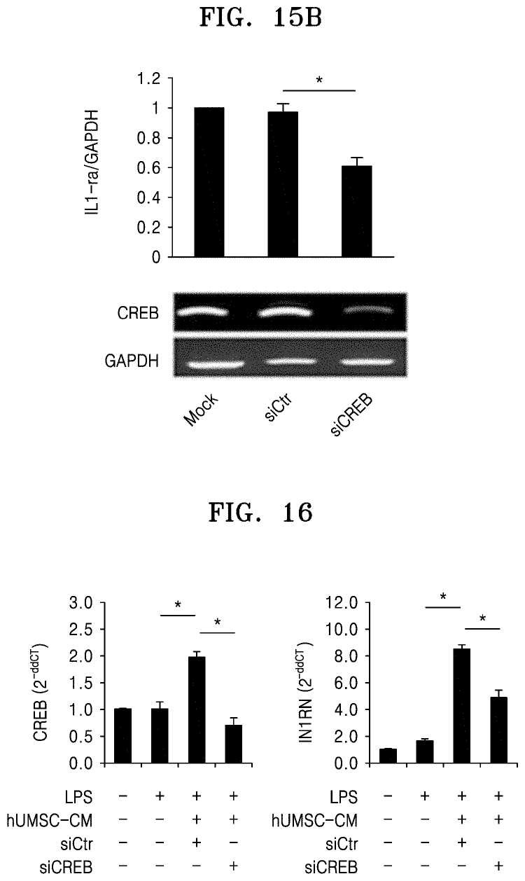

Meanwhile, as shown in FIGS. 14 and 15, when hUMSCs-CM was co-treated with CREB inhibitor (KG501), the increase of IL-1ra expression by hUMSCs-CM treatment was inhibited in a CREB inhibitor dose-dependent manner, and transfection with CREB siRNA (siCREB) also reduced the expression of IL-1 ra protein in Raw 264.7 cells. Further, as shown in FIG. 16, in the LPS-stimulated Raw 264.7 cells, knockdown of CREB reduced the enhanced expression of IL1RN, which was induced by treatment with hUMSCs-CM.

These results of a series of experiments indicate that increased expression of IL-1ra in inflammatory cells including macrophages may contribute to treatment of stroke, and this expression is mediated by CREB.

A composition according to an aspect may include an interleukin-1 (IL-1) receptor antagonist, and administration of the composition in the acute phase of stroke may greatly contribute to recovery of nerve damage and improvement of functions. Therefore, this composition may be usefully applied to the treatment of stroke.

A method of screening according to another aspect may be used to develop a stroke therapeutic agent having excellent therapeutic effects, based on key molecular mechanisms.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A method for treating stroke, the method comprising:
administering a pharmaceutical composition comprising an interleukin-1 (IL-1) receptor antagonist to a subject in need thereof.

2. The method of claim 1, wherein the IL-1 receptor antagonist is umbilical cord-derived mesenchymal stromal cells, interleukin-1 receptor antagonist (IL-1RA) protein, or a combination thereof.

3. The method of claim 1, wherein the IL-1 receptor antagonist is an antibody, an aptamer, an antisense nucleotide against IL-1, or a combination thereof.

4. The method of claim 1, wherein the composition acts on inflammatory cells in lesion tissues.

5. The method of claim 4, wherein the composition increases IL-1RA expression of inflammatory cells in lesion tissues.

6. The method of claim 1, wherein the inflammatory cells are microglia or macrophages.

7. A method of screening for a therapeutic agent for stroke, the method comprising:
measuring an expression level of an interleukin-1 receptor antagonist (IL-1RA) in a sample from a subject which is in contact with a candidate for treating stroke; and
comparing the measured expression level of IL-1RA with an expression level of IL-1RA in a sample from a control subject which is not in contact with the candidate.

8. The method of claim 7, further comprising:
determining the candidate as a therapeutic agent for stroke, when the measured expression level of IL-1RA is increased as compared with the expression level of IL-1RA of the non-contacted control.

9. The method of claim 7, further comprising:
measuring an activity level of cAMP-response element-binding (CREB) protein in a sample from a subject which is in contact with a candidate for treating stroke; and
comparing the measured activity level of CREB protein with an activity level of CREB protein in a sample from a non-contacted control subject.

10. The method of claim 9, further comprising:
determining the candidate as a therapeutic agent for stroke, when the measured activity level of CREB protein is increased as compared with the activity level of CREB protein of the non-contacted control.

11. The method of claim 7, wherein the sample is brain tissue or inflammatory cells isolated from the tissue.

12. The method of claim 11, wherein the inflammatory cells are microglia, macrophages, or a combination thereof.

13-15. (canceled)
